(19) United States
(12) Patent Application Publication
(10) Pub. No.: US 2022/0146519 A1
(43) Pub. Date: May 12, 2022

(54) INTEGRIN ALPHA V BETA 3 TARGETING PROBE FOR DIAGNOSING RETINOCHOROIDAL NEOVASCULAR DISEASES AND PREPARATION METHOD THEREFOR

(71) Applicant: SEOUL NATIONAL UNIVERSITY HOSPITAL, Seoul (KR)

(72) Inventors: Byung Chul LEE, Seongnam-si Gyeonggi-do (KR); Jae Ho JUNG, Seongnam-si Gyeonggi-do (KR); Se Joon WOO, Seongnam-si Gyeonggi-do (KR); Seong Joon AHN, Seongnam-si Gyeonggi-do (KR)

(21) Appl. No.: 17/438,412

(22) PCT Filed: Nov. 8, 2019
(86) PCT No.: PCT/KR2019/015132

§ 371 Date: Sep. 10, 2021

(30) Foreign Application Priority Data
Mar. 11, 2019 (KR) 10-2019-0027284

(51) Int. Cl. G01N 33/58; A61K 49/00; G01N 33/68

(52) U.S. Cl. CPC G01N 33/582; G01N 2800/164; G01N 33/6893; A61K 49/0056

(57) ABSTRACT

Provided are: an integrin targeting probe, which can be effectively used for the diagnosis or treatment of retinochoroidal neovascularization or age-related macular degeneration by predicting the occurrence and recurrence of retinochoroidal neovascularization before structural changes of retinochoroidal neovascularization occur; and a preparation method therefor. The integrin targeting probe is an integrin αᵥβ₃ targeting probe for diagnosing retinochoroidal neovascular diseases and can comprise a fluorescent material-labeled cyclic RGD peptide, which is completed by conjugating an NH₂-cyclic RGD peptide precursor to a fluorescent material.

### TECHNICAL FIELD

The present invention relates to an integrin targeting probe and a preparation method therefor and, more specifically, an integrin αᵥβ₃ targeting probe for diagnosing retinochoroidal neovascular diseases and a preparation therefor.

### BACKGROUND ART

Age-related macular degeneration (AMD) has been reported to be the leading cause of blindness among the elderly in developed countries. Choroidal neovascularization (CNV), known as the key pathogenesis of wet AMD, is one of the main causes of visual impairment in the disease. Structurally, choroidal neovascularization leads to retinal hemorrhage, photoreceptor degeneration, and macular scar formation.

However, the precise mechanisms of choroidal neovascularization development and the key molecules mediating the angiogenesis have been little known. Furthermore, the current clinical imaging methods of AMD, namely fluorescein angiography and optical coherence tomography (OCT), provide only structural information on disease status or developed choroidal neovascularization. Therefore, the imaging methods of macular degeneration according to conventional art could not inform disease progression nor predict the formation or recurrence of choroidal neovascularization.

Meanwhile, integrin αᵥβ₃ is expressed preferentially on angiogenic blood vessels, whereas its expression level in normal tissue is known to be low (Kumar C C, Armstrong L, Yin Z, et al. (2000) Targeting integrins alpha v beta 3 and alpha v beta 5 for blocking tumor-induced angiogenesis. Adv Exp Med Biol 476:169-180). In addition, integrin αᵥβ₃ is reported to be involved in ocular angiogenesis, which is a key pathological process of choroidal neovascularization (Luna J, Tobe T, Mousa S A, Reilly T M, Campochiaro P A (1996) Antagonists of integrin alpha v beta 3 inhibit retinal neovascularization in a murine model. Lab Invest 75:563-573; Friedlander M, Theesfeld C L, Sugita M, et al. (1996) Involvement of integrins alpha v beta 3 and alpha v beta 5 in ocular neovascular diseases. Proc Natl Acad Sci USA 93:9764-9769). Therefore, for the diagnosis and treatment of choroidal neovascularization, research on an integrin αᵥβ₃ targeting probe optimized for choroidal neovascularization is urgently required.

In general, RGD peptide, which is a peptide in which arginine (R), glycine (G), and aspartic acid (D) are bound, has a high affinity for integrin αᵥβ₃, so has been reported to act as an excellent contrast agent for choroidal neovascularization. (McDonald D M, Choyke P L (2003) Imaging of angiogenesis: from microscope to clinic. Nat Med 9:713-725; Gaertner F C, Kessler H, Wester H J, Schwaiger M, Beer A J (2012) Radiolabelled RGD peptides for imaging and therapy. Eur J Nucl Med Mol Imaging 39 Suppl 1:S126-138; Schottelius M, Laufer B, Kessler H, Wester H J (2009) Ligands for mapping alphavbeta3-integrin expression in vivo. Acc Chem Res 42:969-980).

Accordingly, after much effort and research, the present inventors have developed a novel RGD peptide optimized for the diagnosis and treatment of choroidal neovascularization and have completed the present invention.

### DISCLOSURE OF THE INVENTION

### Technical Problem

An embodiment of the present invention provides an integrin targeting probe, which can be effectively used for the diagnosis or treatment of retinochoroidal neovascularization or age-related macular degeneration by predicting the occurrence and recurrence of retinochoroidal neovascularization before structural changes of retinochoroidal neovascularization occur.

An embodiment of the present invention further provides a method for preparing the integrin targeting probe.

However, the present invention is not limited thereto, and other embodiments not mentioned can be clearly understood by those skilled in the art from the following description.

### Technical Solution

An integrin targeting probe according to an embodiment of the present invention is an integrin αᵥβ₃ targeting probe for diagnosing retinochoroidal neovascular diseases and can comprise a fluorescent material-labeled cyclic RGD peptide, which is completed by conjugating an NH₂-cyclic RGD peptide precursor to a fluorescent material.

The NH₂-cyclic RGD peptide precursor is NH₂-D-[c(RGDfK)]₂, and the fluorescent material-labeled cyclic RGD peptide may be FITC-D-[c(RGDfK)]₂.

The fluorescent material may consist of one or more materials selected from the group consisting of fluorescein isothiocyanate (FITC), coumarine, cascade blue, pacific blue, pacific orange, lucifer yellow, NBD, PE, PE-Cy5, PE-Cy7, Red 613, PerCP, TruRed, FluorX, BODIPY-FL, cyanine-based fluorescent materials (Cy2, Cy3, Cy3B, Cy3.5, Cy5, Cy5.5, Cy7), tetramethylrhodamine isothiocyanate (TRITC), X-rhodamine, lissamine rhodamine B, texas red, fluorescein, indocyanine green, and allophycocyanin (APC).

The fluorescent material-labeled cyclic RGD peptide may be used for fluorescence fundus angiography.

A method for preparing an integrin targeting probe according to an embodiment of the present invention is a method for preparing an integrin αᵥβ₃ targeting probe for diagnosing retinochoroidal neovascular diseases and can comprise a step for synthesizing an NH₂-cyclic RGD peptide precursor and a step for conjugating the synthesized NH₂-cyclic RGD peptide precursor to a fluorescent material to complete a fluorescent material-labeled cyclic RGD peptide.

Specific details of other embodiments are included in the detailed description and drawings.

### Effects of the Invention

As described above, the integrin targeting probe of the present invention, that is, the FITC-labeled cyclic RGD peptide, can visualize retinochoroidal neovascularization, the main cause of age-related macular degeneration, and thus allows prediction of the occurrence and recurrence of retinochoroidal neovascularization before structural changes of retinochoroidal neovascularization occur. In particular, it was found that retinochoroidal neovascularization lesions showed intense immunofluorescence staining for the FITC-labeled cyclic RGD peptide of the present invention, unlike the normal retina and choroid. In addition, it was found that normal vessels in the retina were barely stained with the FITC-labeled cyclic RGD peptide of the present invention.

### BRIEF DESCRIPTION OF THE DRAWINGS

FIG. 1 shows images depicting the characterization of choroidal neovascularization formation according to the present invention.

FIG. 2 shows choroidal flatmount immunofluorescence images obtained at 7 days after choroidal neovascularization induction according to the present invention.

FIG. 3 shows the co-localization of the RGD-binding protein with integrin αᵥβ₃.

FIG. 4(a) shows the integrin mRNA expression by reverse transcription-polymerase chain reaction (RT-PCR) in the retina with laser-induced choroidal neovascularization at 1, 3, 7, and 14 days. FIG. 4(b) shows the integrin expression data normalized to the expression of GAPDH gene.

### MODE FOR CARRYING OUT THE INVENTION

Advantages and features of the present invention and methods of achieving the advantages and features will be clear with reference to embodiments described in detail below together with the accompanying drawings. However, the present invention is not limited to embodiments disclosed herein, but will be implemented in various forms. The embodiments are provided so that the present invention is completely disclosed, and a person of ordinary skilled in the art can fully understand the scope of the present invention. Therefore, the present invention will be defined only by the scope of the appended claims. Like reference numerals refer to like elements throughout the specification.

Retinochoroidal neovascular diseases, as mentioned in the present invention, may include, for example, age-related macular degeneration, diabetic retinopathy, retinal vein occlusion, myopic macular degeneration, etc.

In addition, the fluorescent material used for the fluorescent material-labeled cyclic RGD peptide of the present invention may consist of one or more materials selected from the group consisting of fluorescein isothiocyanate (FITC), coumarine, cascade blue, pacific blue, pacific orange, lucifer yellow, NBD, PE, PE-Cy5, PE-Cy7, Red 613, PerCP, TruRed, FluorX, BODIPY-FL, cyanine-based fluorescent materials (Cy2, Cy3, Cy3B, Cy3.5, Cy5, Cy5.5, Cy7), tetramethylrhodamine isothiocyanate (TRITC), X-rhodamine, lissamine rhodamine B, texas red, fluorescein, indocyanine green, and allophycocyanin (APC). However, the present invention is not limited thereto, and any fluorescent material capable of increasing the fluorescence level of a target may be used. In the present invention, experimentation was conducted using FITC, which is suitable for visualizing retinochoroidal neovascularization, as an example.

### Example 1. Preparation of Animals and Materials

<1-1> Preparation of Mice

All mouse model research used for choroidal neovascularization was approved by the Institutional Animal Care and Use Committee of the Seoul National University Hospital and adhered to the Association for Research in Vision and Ophthalmology (ARVO) statement for the Use of Animals in Ophthalmic and Vision Research. In total, 29 wild-type 6-week-old C57BL/6 male mice weighing 22 to 25 g were used for the experiments.

<1-2> Induction of Choroidal Neovascularization

Choroidal neovascularization was induced as follows, according to the literature (Reich S J, Fosnot J, Kuroki A, et al. (2003) Small interfering RNA (siRNA) targeting VEGF effectively inhibits ocular neovascularization in a mouse model. Mol Vis 9:210-216). After intravenous anesthesia using a 1:1 mixture of 100 mg/mL ketamine and 20 mg/mL xylazine and pupillary dilatation using 5.0% phenylephrine and 0.8% tropicamide, C57BL/6 mice were placed on the Mayo stand (Coherent PC-920 Argon Ion Laser System; Coherent Medical Laser, Santa Clara, Calif.). Choroidal neovascularization was induced using 512-nm argon laser photocoagulation, with 100 urn of spot size and 100 mW of power for 0.1 s in the right eye. Five lesions of about 2-3 disc diameters were generated from the optic disc. The formation of bubbles upon laser delivery can be considered sufficient damage to induce rupture of Bruch's membrane and choroidal neovascularization. When subretinal hemorrhage occurred after the laser treatment, the mice were excluded from the experiment.

<1-3> Preparation of FITC-Labeled Cyclic RGD Peptide

The cyclic RGD peptide was synthesized from the protected cyclic RGD peptide, i.e., cyclic R(Pdf)-G-D(tBu)-f-K—NH₂, purchased from Bio Imaging Korea Co., Ltd. (R=arginine; Pdf=pentamethylbenzofuransulfonyl; G=glycine; D=aspartic acid; tBu=tert-butyl; f=D-phenylalanine, K=lysine). The starting material, cyclic R(Pdf)-G-D(tBu)-f-K—NH₂ (0.4 mmol), N-hydroxybenzotriazole (0.46 mmol), and O-benzotriazole-N,N,N′,N′-tetramethyl-uronium-hexafluorophosphate (0.46 mmol) were added to Boc-protected aspartic acid (0.12 mmol) dissolved in N,N′-dimethylformamide (5 mL) under nitrogen gas atmosphere and were stirred at room temperature for 12 hours. The solvent was removed under reduced pressure. Then, column chromatography was performed to obtain a compound, in which the cyclic RGD dimer peptide, BocNH-D-[c(R(Pdf)-G-D(tBu)-f-K)]₂ (MS (ESI) m/z=2020.4 (M+H)⁺), was introduced into aspartic acid. Subsequently, to remove the protecting group, the compound was dissolved in TFA:Et₃SiH:H₂O (95:2.5:2.5, 3 mL) and allowed to react at room temperature for 6 hours. Then, all the solutions were almost evaporated under reduced pressure. Then, diethyl ether was added and the resulting solid was filtered. Thus obtained white solid was sufficiently washed with ester and dried to prepare the NH₂-cyclic RGD peptide precursor, NH₂-D-[c(RGDfK)]₂ (MS (ESI) m/z=1304.2 (M+H)⁺). The obtained NH₂-D-[c(RGDfK)]₂ (10 nmol) was conjugated through urea linkage to 4 μg of fluorescein isothiocyanate (FITC; Thermo Fisher Scientific Korea Inc., Seoul, Korea) in 100 mM phosphate buffered solution (PBS, pH 7.5) with stirring for 1 hour at room temperature. The FITC-labeled peptide, FITC-D-[c(RGDfK)]₂, was purified to at least 95% purity using C-18 reverse phase high-performance liquid chromatography (HPLC; Shimadzu Prominence, Kyoto, Japan) with a solvent mixture of acetonitrile/water/0.1% trifluoroacetic acid and confirmed using mass spectrometry (HP/Agilent 1100 series LC/MSD, Santa Clara, Calif., USA) (MS (ESI) m/z=1693.3 (M+H)⁺).

### Example 2. Histological and Angiographic Evaluation

Mice were euthanized and the eyes were enucleated and fixed in 4% paraformaldehyde. Serial sections of six eyes, enucleated at 2 weeks following choroidal neovascularization induction, were cut at 20 um of thickness on a cryostat (HM550MP; Thermo Scientific, Waltham, Mass., USA) at −20° C., and prepared for staining. Hematoxylin and eosin (H&E) staining was performed for histological examination of the retina and choroid.

Ten eyes were prepared as choroidal flatmounts. For the flatmounts, mice were anesthetized at days 7 or 14 and eyes were enucleated and fixed with 4% paraformaldehyde for 30 min at 4° C. The anterior segment and retina were removed from the eyecup, and four radial incisions were made. The remaining retinal pigment epithelium (RPE)-choroid-sclera complex was flatmounted and coverslipped. Flatmounts were examined with a scanning laser confocal microscope (LSM710; Carl Zeiss, Oberkochen, Germany).

Fluorescein angiography (FA) was performed using a commercial fundus camera and an imaging system (Heidelberg Retina Angiography, Heidelberg Engineering, Heidelberg, Germany) following intraperitoneal injection of 0.2 mL of 2% fluorescein sodium at 1 week after laser photocoagulation. Choroidal neovascularization was confirmed with fluorescein angiography as a hyperfluorescent lesion with late-phase leakage.

### Example 3. Fluorescence Staining of Vessels in Retinal and Choroidal Flatmounts Using RGD Peptides

The enucleated eyes were fixed in 2% paraformaldehyde/PBS (pH 7.4) for 5 min The retina and choroid were then isolated from eyeballs and permeabilized with 0.5% Triton X-100, 5% fetal bovine serum, and 20% dimethyl sulfoxide (DMSO) in PBS for 3 hours at room temperature. For vessel staining, the retinas were incubated with BS-1 lectin-TRITC (Sigma-Aldrich) at 4° C. for 4 days. The earlier prepared FITC-labeled cyclic RGD peptide, FITC-D-[c(RGDfK)]₂, was used for integrin αᵥβ₃ targeting in choroidal neovascularization lesions.

Fluorescence staining with FITC-D-[c(RGDfK)]² was performed as follows:
(1) the retinal and choroidal flatmounts were washed with PBS and incubated with FITC-D-[c(RGDfK)]₂ for 30 minutes;
(2) the slides were washed with PBS several times, counterstained with 4′,6-diamidino-2-phenylindole (DAPI), and mounted with ProLong Gold anti-fade reagent (Life Technologies, Carlsbad, Calif., USA);
(3) and after staining, the flatmounts were mounted with the vitreous side up on glass slides and visualized on a confocal microscope (LSM710; Carl Zeiss, Oberkochen, Germany).

Additionally, the specificity of FITC-D-[c(RGDfK)]₂ staining was evaluated by using an excess of cRGD peptides. For this experiment, one mouse with identical laser-induced choroidal neovascularization in both eyes was sacrificed. One eye of the mouse was stained with the staining method described above using 10 nM of FITCD-[c(RGDfK)]₂. The other eye was stained with the staining method described above plus a 2-hour incubation with excess cRGD peptides (for example, 20-fold molar concentration of the FITC-conjugated cRGD dimer, i.e., 200 nM) prior to the fluorescence staining. In this staining, the present inventors used an integrin αᵥβ₃ antibody to investigate if the staining with the integrin αᵥβ₃ antibody co-localized with that of the FITC-conjugated cRGD dimer.

### Example 4. RT-PCR for Integrin Expression In Vitro

At baseline and at 1, 3, 7, and 14 days after choroidal neovascularization induction, four mice per time point were sacrificed and their eyeballs were enucleated. Total RNA was isolated from the retinal tissue using the RNeasy mini kit (BioRad, Hercules, Calif., USA). Reverse transcription (RT) was performed on 2 μg denatured RNA using the Superscript III First-strand Synthesis kit (Invitrogen). The relative abundance of integrins was analyzed using semi-quantitative polymerase chain reaction (PCR) with BioMix (Bioline, London, UK) according to the manufacturer's protocol. Negative controls were performed without RT to confirm the absence of genomic DNA contamination. The reaction conditions of the above sequences were as follows: denaturation at 95° C. for 5 minutes, extension at 58° C. for 45 seconds, and annealing at 72° C. for 60 seconds for 33 cycles. PCR products were separated on a 3% agarose gel by electrophoresis for 20 minutes at 150 V. PCR products were identified by their expected size.

### Reference Example 1. Statistical Analysis

The Wilcoxon signed rank test was used to assess differences among paired groups. Mann-Whitney test was used for comparison between independent groups. Continuous values are expressed as mean±standard error (SE). P values less than 0.05 were considered statistically significant. Statistical analyses were performed by using SPSS version 18.0 (SPSS Inc., Chicago, Ill., USA).

### Experimental Example 1. Confirmation of Choroidal Neovascularization Formation

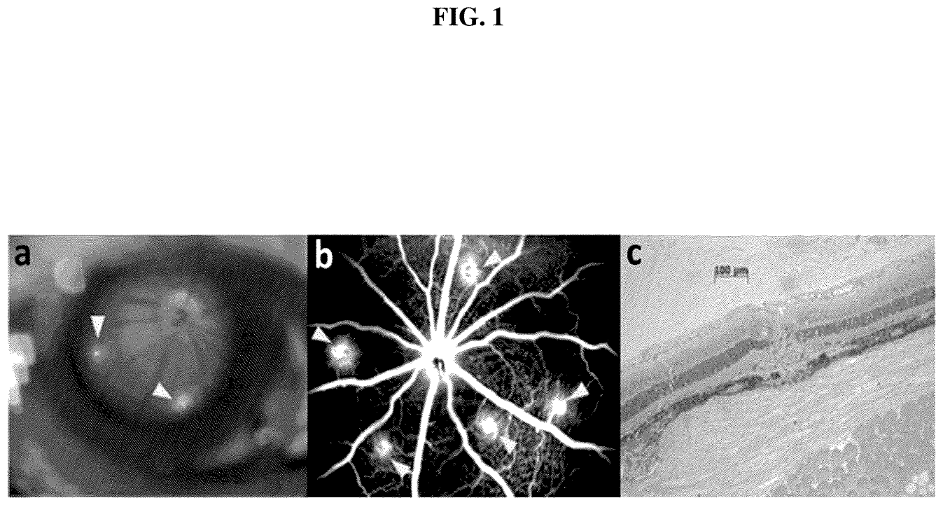

FIG. 1 shows images depicting the characterization of choroidal neovascularization formation according to the present invention. (a) of FIG. 1 is the fundus image obtained immediately after laser photocoagulation (arrowheads indicate the laser-treated spots. Bubble formation is noted immediately after Bruch's membrane rupture). (b) of FIG. 1 shows the choroidal neovascularization lesions with dye leakage from the laser-treated spots (arrowheads) by fluorescein angiography. (c) of FIG. 1 shows hematoxylin and eosin (H&E)-stained cryosection at 2 weeks following choroidal neovascularization induction.

Specifically, as depicted in (a) of FIG. 1, choroidal neovascularization was induced by laser photocoagulation and Bruch's membrane was disrupted. Immediately after laser induction, vaporization bubbles formed. As depicted in (b) of FIG. 1, the formation of choroidal neovascularization was confirmed using fluorescein angiography (FA). Fluorescein angiography revealed hyperfluorescent spots with fluorescein leakage at the areas in which laser photocoagulation was performed, which is compatible with choroidal neovascularization. The spots with leakage were matched with those treated by laser induction.

As depicted in (c) of FIG. 1, histopathologically, choroidal neovascularization-induced eyes showed fibrovascular complex formation in the choroid and the retina with disruption of the retinal pigment epithelium (RPE) and the outer retina, which is compatible with choroidal neovascularization.

### Experimental Example 2. Ex Vivo Imaging of Choroidal Neovascularization and Co-Localization of Integrins

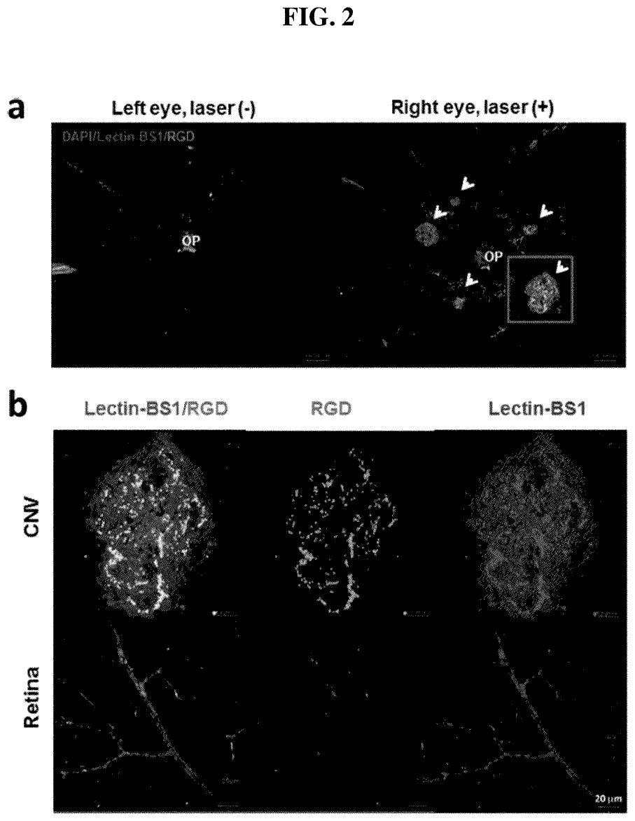

FIG. 2 shows choroidal flatmount immunofluorescence images obtained at 7 days after choroidal neovascularization induction according to the present invention. In (a) of FIG. 2, compared to the untreated eye (left), the eye with choroidal neovascularization induction (right) shows hyperfluorescent spots, which correspond to the laser-treated areas (arrowheads). (b) of FIG. 2 shows enlarged images of choroidal neovascularization (square in (a) of FIG. 2), which show that the laser-treated lesion stained with FITC-labeled RGD peptide corresponded to that stained with lectin, indicating that choroidal neovascularization can be stained with an RGD-based probe. In the untreated retina (bottom), FITC-labeled RGD peptide immunofluorescence is observed along the retinal vessels (OP=optic disc).

Specifically, as depicted in FIG. 2, the FITC-labeled cyclic RGD peptide allowed the visualization of choroidal neovascularization at laser-treated areas. In particular, as depicted in (a) of FIG. 2, compared to the untreated eye ((a) of FIG. 2, left), the choroidal neovascularization-related eye showed five lectin-positive, RGD peptide-binding spots (arrowheads). These spots topographically matched with the five laser-treated spots ((a) of FIG. 2, right). These spots were co-stained with DAPI, lectin, and RGD peptide. As depicted in (b) of FIG. 2, an enlarged image of one of the spots better demonstrates the co-localization of the RGD-binding protein (integrin αᵥβ₃) with lectin ((b) of FIG. 2, top). In contrast, normal vessels in the retina, which are lectin-positive, were barely stained with FITC-labeled RGD peptide ((b) of FIG. 2, bottom). This indicates that when an RGD peptide dimer-integrated probe binds to choroidal neovascularization, choroidal neovascularization can be imaged using the RGD peptide dimer-integrated probe.

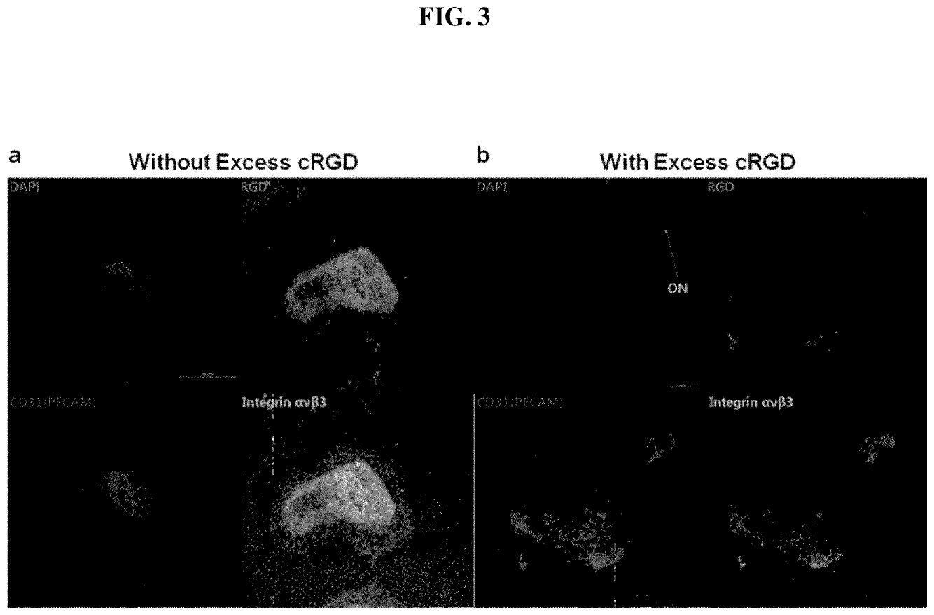

FIG. 3 shows the co-localization of the RGD-binding protein with integrin αᵥβ₃. Specifically, FIG. 3 shows fluorescence staining of choroidal flatmounts in a mouse treated with laser identically in both eyes. Both eyes were co-stained with DAPI, FITC-RGD, CD31, and integrin αᵥβ₃ antibody. The left eye (b) was incubated with excess cRGD before FITC-RGD staining. In this instance, it was found that the fluorescence of the FITC-labeled RGD peptide was remarkably reduced by excess cRGD.

The images of FITC-D-[c(RGDfK)]₂ angiography depicted in FIG. 3 have higher resolution than those of conventional SPECT using radioisotopes.

### Experimental Example 3. Integrin mRNA Expression Following Choroidal Neovascularization Induction

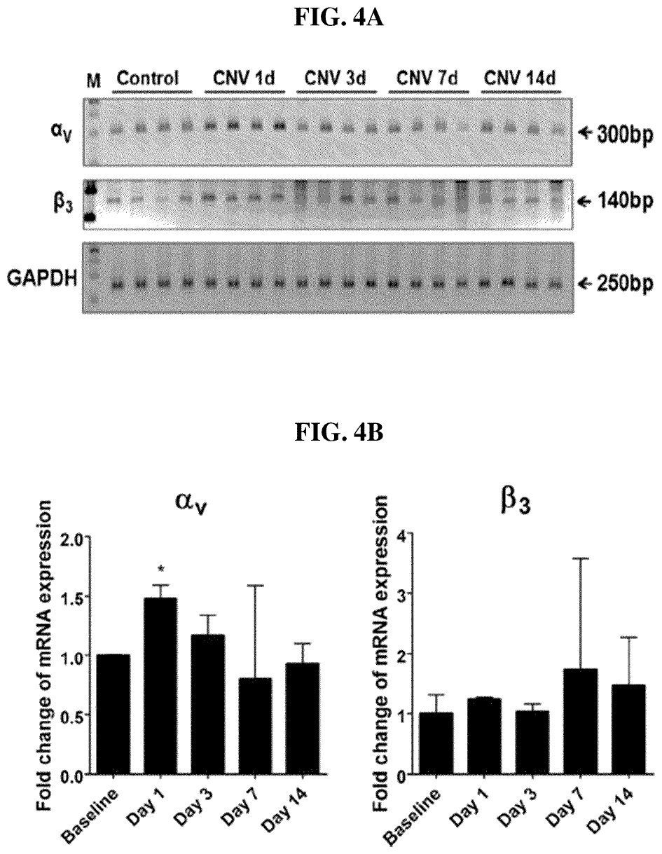

FIG. 4(a) shows the integrin mRNA expression by reverse transcription-polymerase chain reaction (RT-PCR) in the retina with laser-induced choroidal neovascularization at 1, 3, 7, and 14 days. FIG. 4(b) shows the integrin expression data normalized to the expression of GAPDH gene. Upper bars indicate upper bound of 95% confidence interval (P<0.05).

Specifically, as depicted in FIG. 4, the integrin expression was examined using RT-PCR in the mouse retina over time following choroidal neovascularization induction. When normalized to the expression of the glyceraldehyde-3-phosphate dehydrogenase (GAPDH) gene, most integrins showed similar pattern of expression. That is, as depicted in FIG. 4(b), the pattern of an increase at the early stages (peak at day 1) and a subsequent decrease over the following 2-week period to the level similar to that at baseline was exhibited. There was an increase in the expression of integrin αᵥ (1.48-fold) and β₃ (1.24-fold) at day 1 after choroidal neovascularization induction. The increase in the expression of integrin αᵥ at day 1 was statistically significant (P<0.05). At days 3, 7, and 14, there were no significant changes in the expression of integrin αᵥ or β₃ compared to baseline.

Although the embodiments of the present invention have been described above with reference to the accompanying drawings, it will be understood by those skilled in the art to which the present invention belongs that the present invention can be implemented in other specific forms without changing the technical spirit or essential features thereof. It is, therefore, to be understood that the above-described embodiments are illustrative in all aspects and not restrictive.

1. An integrin targeting probe, which is an integrin αᵥβ₃ targeting probe for diagnosing retinochoroidal neovascular diseases and comprises a fluorescent material-labeled cyclic RGD peptide which is completed by conjugating an NH₂-cyclic RGD peptide precursor to a fluorescent material.

2. The integrin targeting probe of claim 1, wherein the NH₂-cyclic RGD peptide precursor is NH₂-D-[c(RGDfK)]₂, and the fluorescent material-labeled cyclic RGD peptide is FITC-D-[c(RGDfK)]₂.

3. The integrin targeting probe of claim 1, wherein the fluorescent material consists of one or more materials selected from the group consisting of fluorescein isothiocyanate (FITC), coumarin, cascade blue, pacific blue, pacific orange, lucifer yellow, NBD, PE, PE-Cy5, PE-Cy7, Red 613, PerCP, TruRed, FluorX, BODIPY-FL, cyanine-based fluorescent materials (Cy2, Cy3, Cy3B, Cy3.5, Cy5, Cy5.5, Cy7), tetramethylrhodamine isothiocyanate (TRITC), X-rhodamine, lissamine rhodamine B, texas red, fluorescein, indocyanine green, and allophycocyanin (APC).

4. The integrin targeting probe of claim 1, wherein the fluorescent material-labeled cyclic RGD peptide is used for fluorescence fundus angiography.

5. A method for preparing an integrin targeting probe, which is a method for preparing an integrin αᵥβ₃ targeting probe for diagnosing retinochoroidal neovascular diseases and comprises a step for synthesizing an NH₂-cyclic RGD peptide precursor and a step for conjugating the synthesized NH₂-cyclic RGD peptide precursor to a fluorescent material to complete a fluorescent material-labeled cyclic RGD peptide.

6. The method for preparing an integrin targeting probe of claim 5, wherein the NH₂-cyclic RGD peptide precursor is NH₂-D-[c(RGDfK)]₂, and the fluorescent material-labeled cyclic RGD peptide is FITC-D-[c(RGDfK)]₂.

7. The method for preparing an integrin targeting probe of claim 5, wherein the fluorescent material consists of one or more materials selected from the group consisting of fluorescein isothiocyanate (FITC), coumarine, cascade blue, pacific blue, pacific orange, lucifer yellow, NBD, PE, PE-Cy5, PE-Cy7, Red 613, PerCP, TruRed, FluorX, BODIPY-FL, cyanine-based fluorescent materials (Cy2, Cy3, Cy3B, Cy3.5, Cy5, Cy5.5, Cy7), tetramethylrhodamine isothiocyanate (TRITC), X-rhodamine, lissamine rhodamine B, texas red, fluorescein, indocyanine green, and allophycocyanin (APC).

8. The method for preparing an integrin targeting probe of claim 5, wherein the fluorescent material-labeled cyclic RGD peptide is used for fluorescence fundus angiography.
